(19) United States
(12) Patent Application Publication
(10) Pub. No.: US 2020/0402623 A1
(43) Pub. Date: Dec. 24, 2020

(54) SYSTEM AND METHODS FOR RXTOKEN ECONOMICS

(71) Applicants: Underland Carl, Philadelphia, PA (US); Ugur Amet, Philadelphia, PA (US)

(72) Inventors: Underland Carl, Philadelphia, PA (US); Ugur Amet, Philadelphia, PA (US)

(21) Appl. No.: 16/857,165

(22) Filed: Apr. 23, 2020

Related U.S. Application Data
(60) Provisional application No. 62/837,391, filed on Apr. 23, 2019.

(51) Int. Cl. G16H 10/60; H04L 9/06; G06Q 30/02

(52) U.S. Cl. CPC G16H 10/60; H04L 2209/38; G06Q 30/0207; H04L 9/0637

(57) ABSTRACT

The present disclosure is directed to blockchain smart contracts between a captive insurer and a health provider or associate wherein RxTokens are transferred by the captive insurer when one of the agreed upon smart contract terms are satisfied.

### CROSS REFERENCE TO RELATED APPLICATIONS

This application claims the benefit of U.S. Provisional Application No. 62/837,391 filed on Apr. 23, 2019, the contents of which are incorporated by reference herein.

### FIELD OF THE INVENTION

The present invention relates to a decentralized data structure, and, more particularly, a data structure that maintains a continuously growing list of records, or blocks, in a blockchain format in the realm of blockchain enabled health insurance.

### BACKGROUND

The current status of health insurance involves a financial risk model where an insurer has no incentive to define & track health outcomes over time, so members have no expectation that system incentives somehow tie in with their own prevailing health status. Also, this current health insurance model provides no reasons to incentivize health providers to promote member health over financial gain.

### BRIEF DESCRIPTION OF THE DRAWINGS

This disclosure is illustrated by way of example and not by way of limitation in the accompanying figure(s). The figure(s) may, alone or in combination, illustrate one or more embodiments of the disclosure. Elements illustrated in the figure(s) are not necessarily drawn to scale. Reference labels may be repeated among the figures to indicate corresponding or analogous elements.

The detailed description makes reference to the accompanying figures in which:

FIG. 1 is a block diagram of an exemplary computing system for use in accordance with herein described systems and methods;

FIG. 2 is a block diagram showing an exemplary networked computing environment for use in accordance with herein described systems and methods;

FIG. 3 is an illustration of a distributed ledger for use in the present invention;

FIG. 4 is an illustration of a distributed ledger for use in the present invention;

FIG. 5 is an illustration of a distributed ledger use environment;

FIG. 6 is an illustration of an exemplary networked system environment for use in accordance with herein described systems and methods;

FIG. 7 is an illustration of a blockchain environment for use in the present invention;

FIG. 8 is an illustration of an exemplary smart contract for use in accordance with herein described systems and methods;

FIG. 9 is an illustration of an exemplary process flow for use in accordance with herein described systems and methods; and

FIG. 10 is a block diagram showing an exemplary networked computing environment for use in accordance with herein described systems and methods.

### DETAILED DESCRIPTION

The figures and descriptions provided herein may have been simplified to illustrate aspects that are relevant for a clear understanding of the herein described apparatuses, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

Embodiments are provided throughout so that this disclosure is sufficiently thorough and fully conveys the scope of the disclosed embodiments to those who are skilled in the art. Numerous specific details are set forth, such as examples of specific components, devices, and methods, to provide a thorough understanding of embodiments of the present disclosure. Nevertheless, it will be apparent to those skilled in the art that certain specific disclosed details need not be employed, and that exemplary embodiments may be embodied in different forms. As such, the exemplary embodiments should not be construed to limit the scope of the disclosure. As referenced above, in some exemplary embodiments, well-known processes, well-known device structures, and well-known technologies may not be described in detail.

Components, or modules, shown in diagrams are illustrative of exemplary embodiments of the invention and are meant to avoid obscuring the invention. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components. Components may be implemented in software, hardware, or a combination thereof.

Furthermore, connections between components or systems within the figures are not intended to be limited to direct connections. Rather, data between these components may be modified, re-formatted, or otherwise changed by intermediary components. Also, additional or fewer connections may be used. It shall also be noted that the terms “coupled,” “connected,” or “communicatively coupled” shall be understood to include direct connections, indirect connections through one or more intermediary devices, and wireless connections.

The use of certain terms in various places in the specification is for illustration and should not be construed as limiting. A service, function, or resource is not limited to a single service, function, or resource; usage of these terms may refer to a grouping of related services, functions, or resources, which may be distributed or aggregated.

The terms “messages,” “blocks,” and “data,” shall be understood to mean a group of bits, which may be transported across a network. These terms shall not be interpreted as limiting embodiments of the present invention to particular configuration; and, these terms along with similar terms such as “data,” “data traffic,” “information,” “cell,” etc. may be replaced by other terminologies referring to a group of bits, and may be used interchangeably. The terms “include,” “including,” “comprise,” and “comprising” shall be understood to be open terms and any lists the follow are examples and not meant to be limited to the listed items. Any headings used herein are for organizational purposes only and shall not be used to limit the scope of the description or the claims.

The terminology used herein is for the purpose of describing particular exemplary embodiments only and is not intended to be limiting. For example, as used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The steps, processes, and operations described herein are not to be construed as necessarily requiring their respective performance in the particular order discussed or illustrated, unless specifically identified as a preferred or required order of performance. It is also to be understood that additional or alternative steps may be employed, in place of or in conjunction with the disclosed aspects.

When an element or layer is referred to as being “on,” “engaged to,” “connected to,” or “coupled to” another element or layer, it may be directly on, engaged, connected or coupled to the other element or layer, or intervening elements or layers may be present, unless clearly indicated otherwise. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). Further, as used herein the term “and/or” includes any and all combinations of one or more of the associated listed items.

Yet further, although the terms first, second, third, etc. may be used herein to describe various elements, components, regions, layers and/or sections, these elements, components, regions, layers and/or sections should not be limited by these terms. These terms may be only used to distinguish one element, component, region, layer or section from another element, component, region, layer or section. Terms such as “first,” “second,” and other numerical terms when used herein do not imply a sequence or order unless clearly indicated by the context. Thus, a first element, component, region, layer or section discussed below could be termed a second element, component, region, layer, or section without departing from the teachings of the exemplary embodiments.

A computer-implemented platform and methods of use are disclosed that provide networked access to a plurality of types of digital content, including but not limited to video, audio, and document content, and that track and deliver the accessed content, such as via one or more applications, or “apps.” Described embodiments are intended to be exemplary and not limiting. As such, it is contemplated that the herein described systems and methods can be adapted to provide many types of users with access and delivery of many types of domain data, and can be extended to provide enhancements and/or additions to the exemplary services described. The invention is intended to include all such extensions. Reference will now be made in detail to various exemplary and illustrative embodiments of the present invention.

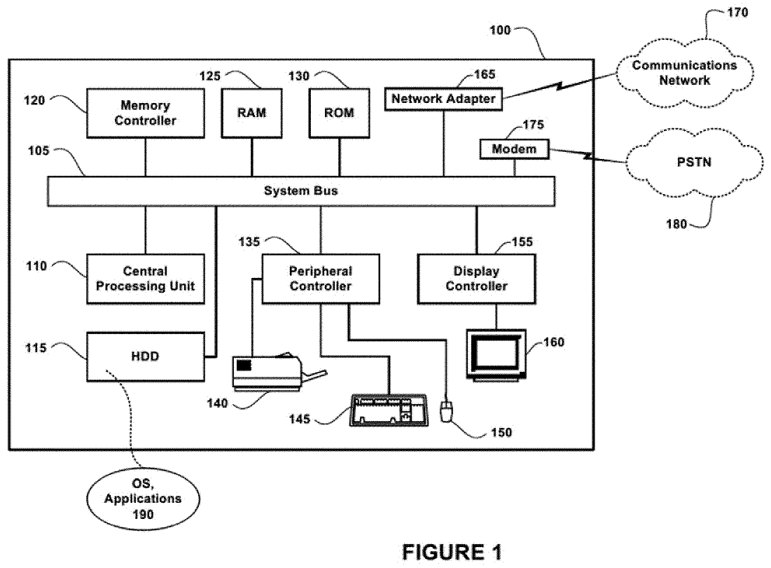

FIG. 1 depicts an exemplary computing system 100 that can be used in accordance with herein described system and methods. Computing system 100 is capable of executing software, such as an operating system (OS) and a variety of computing applications 190. The operation of exemplary computing system 100 is controlled primarily by computer readable instructions, such as instructions stored in a computer readable storage medium, such as hard disk drive (HDD) 115, optical disk (not shown) such as a CD or DVD, solid state drive (not shown) such as a USB “thumb drive,” or the like. Such instructions may be executed within central processing unit (CPU) 110 to cause computing system 100 to perform operations. In many known computer servers, workstations, personal computers, mobile devices, and the like, CPU 110 is implemented in an integrated circuit called a processor.

It is appreciated that, although exemplary computing system 100 is shown to comprise a single CPU 110, such description is merely illustrative as computing system 100 may comprise a plurality of CPUs 110. Additionally, computing system 100 may exploit the resources of remote CPUs (not shown), for example, through communications network 170 or some other data communications means.

In operation, CPU 110 fetches, decodes, and executes instructions from a computer readable storage medium such as HDD 115. Such instructions can be included in software such as an operating system (OS), executable programs, and the like. Information, such as computer instructions and other computer readable data, is transferred between components of computing system 100 via the system's main data-transfer path. The main data-transfer path may use a system bus architecture 105, although other computer architectures (not shown) can be used, such as architectures using serializers and deserializers and crossbar switches to communicate data between devices over serial communication paths. System bus 105 can include data lines for sending data, address lines for sending addresses, and control lines for sending interrupts and for operating the system bus. Some busses provide bus arbitration that regulates access to the bus by extension cards, controllers, and CPU 110. Devices that attach to the busses and arbitrate access to the bus are called bus masters. Bus master support also allows multiprocessor configurations of the busses to be created by the addition of bus master adapters containing processors and support chips.

Memory devices coupled to system bus 105 can include random access memory (RAM) 125 and read only memory (ROM) 130. Such memories include circuitry that allows information to be stored and retrieved. ROMs 130 generally contain stored data that cannot be modified. Data stored in RAM 125 can be read or changed by CPU 110 or other hardware devices. Access to RAM 125 and/or ROM 130 may be controlled by memory controller 120. Memory controller 120 may provide an address translation function that translates virtual addresses into physical addresses as instructions are executed. Memory controller 120 may also provide a memory protection function that isolates processes within the system and isolates system processes from user processes. Thus, a program running in user mode can normally access only memory mapped by its own process virtual address space; it cannot access memory within another process' virtual address space unless memory sharing between the processes has been set up.

In addition, computing system 100 may contain peripheral controller 135 responsible for communicating instructions using a peripheral bus from CPU 110 to peripherals, such as printer 140, keyboard 145, and mouse 150. An example of a peripheral bus is the Peripheral Component Interconnect (PCI) bus.

Display 160, which is controlled by display controller 155, can be used to display visual output generated by computing system 100. Such visual output may include text, graphics, animated graphics, and/or video, for example. Display 160 may be implemented with a CRT-based video display, an LCD-based display, gas plasma-based display, touch-panel, or the like. Display controller 155 includes electronic components required to generate a video signal that is sent to display 160.

Further, computing system 100 may contain network adapter 165 which may be used to couple computing system 100 to an external communication network 170, which may include or provide access to the Internet, and hence which may provide or include tracking of and access to the domain data discussed herein. Communications network 170 may provide user access to computing system 100 with means of communicating and transferring software and information electronically, and may be coupled directly to computing system 100, or indirectly to computing system 100, such as via PSTN or cellular network 180. For example, users may communicate with computing system 100 using communication means such as email, direct data connection, virtual private network (VPN), Skype or other online video conferencing services, or the like. Additionally, communications network 170 may provide for distributed processing, which involves several computers and the sharing of workloads or cooperative efforts in performing a task. It is appreciated that the network connections shown are exemplary and other means of establishing communications links between computing system 100 and remote users may be used.

It is appreciated that exemplary computing system 100 is merely illustrative of a computing environment in which the herein described systems and methods may operate and does not limit the implementation of the herein described systems and methods in computing environments having differing components and configurations, as the inventive concepts described herein may be implemented in various computing environments using various components and configurations.

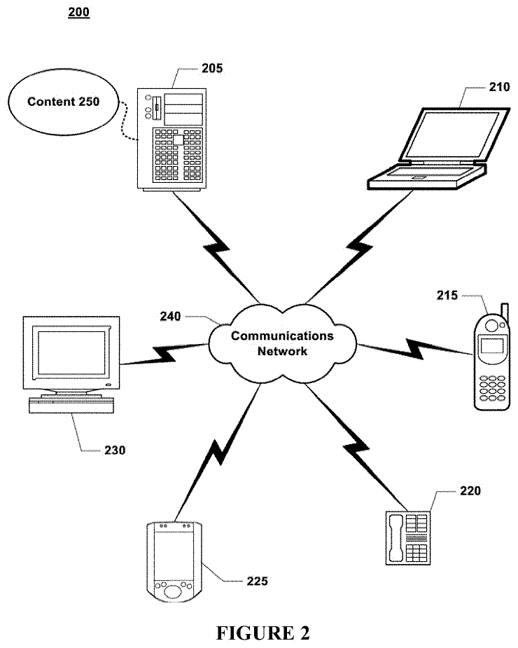

As shown in FIG. 2, computing system 100 can be deployed in networked computing environment 200. In general, the above description for computing system 100 applies to server, client, and peer computers deployed in a networked environment, for example, server 205, laptop computer 210, and desktop computer 230. FIG. 2 illustrates an exemplary illustrative networked computing environment 200, with a server in communication with client computing and/or communicating devices via a communications network, in which the herein described apparatus and methods may be employed.

As shown in FIG. 2, server 205 may be interconnected via a communications network 240 (which may include any of, or any combination of, a fixed-wire or wireless LAN, WAN, intranet, extranet, peer-to-peer network, virtual private network, the Internet, or other communications network such as POTS, ISDN, VoIP, PSTN, etc.) with a number of client computing/communication devices such as laptop computer 210, wireless mobile telephone 215, wired telephone 220, personal digital assistant 225, user desktop computer 230, and/or other communication enabled devices (not shown). Server 205 can comprise dedicated servers operable to process and communicate data such as digital content 250 to and from client devices 210, 215, 220, 225, 230, etc. using any of a number of known protocols, such as hypertext transfer protocol (HTTP), file transfer protocol (FTP), simple object access protocol (SOAP), wireless application protocol (WAP), or the like. Additionally, networked computing environment 200 can utilize various data security protocols such as secured socket layer (SSL), pretty good privacy (PGP), virtual private network (VPN) security, or the like. Each client device 210, 215, 220, 225, 230, etc. can be equipped with an operating system operable to support one or more computing and/or communication applications, such as a web browser (not shown), email (not shown), or independently developed applications, the like, to interact with server 205.

The server 205 may thus deliver applications specifically designed for mobile client devices, such as, for example, client device 225. A client device 225 may be any mobile telephone, PDA, tablet or smart phone and may have any device compatible operating system. Such operating systems may include, for example, Symbian, RIM Blackberry OS, Android, Apple iOS, Windows Phone, Palm webOS, Maemo, bada, MeeGo, Brew OS, and Linux for smartphones and tablets. Although many mobile operating systems may be programmed in C++, some may be programmed in Java and .NET, for example. Some operating systems may or may not allow for the use of a proxy server and some may or may not have on-device encryption. Of course, because many of the aforementioned operating systems are proprietary, in prior art embodiments server 205 delivered to client device 225 only those applications and that content applicable to the operating system and platform communication relevant to that client device 225 type.

JavaScript Serialized Object Notation (JSON), a lightweight, text-based, language-independent data-interchange format, is based on a subset of the JavaScript Programming Language, Standard ECMA-262, 3.sup.rd Edition, dated December 1999. JSON syntax is a text format defined with a collection of name/value pairs and an ordered list of values. JSON is very useful for sending structured data over wire (e.g., the Internet) that is lightweight and easy to parse. It is language and platform independent, but uses conventions that are familiar to C-family programming conventions. The JSON language is thus compatible with a great many operating systems (a list of such systems is available at www.json.org).

The techniques described herein may be used for various wireless communication networks, such as CDMA, TDMA, FDMA, OFDMA, SCFDMA, and other wireless networks. The terms “network” and “system” are often used interchangeably herein. By way of example, a CDMA network may implement a radio technology such as Universal Terrestrial Radio Access (UTRA), cdma2000, and the like. For example, an OFDMA network may implement a radio technology such as Evolved UTRA (E-UTRA), Ultra Mobile Broadband (UMB), IEEE 802.11 (Wi-Fi), IEEE 802.16 (WiMAX), IEEE 802.20, Flash-OFDMÒ, and the like. UTRA and E-UTRA are part of Universal Mobile Telecommunication System (UMTS). UTRA, E-UTRA, UMTS, as well as long term evolution (LTE) and other cellular techniques, are described in documents from an organization named “3rd Generation Partnership Project” (3GPP) and “3rd Generation Partnership Project 2” (3GPP2), such as, for example, the mobile standards for 5G which will begin in the 3GPP Release 15.

“WiFi” stands for “Wireless Fidelity.” WiFi is typically deployed as a wireless local area network (WLAN) that may extend home and business networks to wireless medium. As referenced, the IEEE 802,11 standard defines WiFi communications as between devices, and as between devices and access points. WiFi typically provides aggregate user data speeds from 2 Mbps (for 802.11b) to approximately 150 Mbps (for 802.11n). Typical speeds for WiFi are around 15 Mbps, and latency (i.e., packet delay) averages around 10 ms with no load. WiFi may link devices, and/or devices and access points, over distances from a few feet to several miles. By way of contrast, LTE, as mentioned above, typically provides WAN connectivity that may stretch for much greater distances, but is typically not preferred for LAN communications. Of note, the techniques described herein may be used for the wireless networks and radio technologies mentioned above, as well as for other wireless networks and radio technologies.

WiFi networks, herein also referred to as IEEE 802.11 wireless networks, may operate in two modes: infrastructure mode and ad-hoc mode. In infrastructure mode, a device connects to an access point (AP) that serves as a hub for connecting wireless devices to the network infrastructure, including, for example, connecting wireless devices to Internet access. Infrastructure mode thus uses a client-server architecture to provide connectivity to the other wireless devices. In contrast to the client-server architecture of infrastructure mode, in ad-hoc mode wireless devices have direct connections to each other in a peer-to-peer architecture.

In various embodiments, a blockchain, or distributed ledger, provides a decentralized approach to tracking information. By eliminating the need for a central authority, information and transactions therewith may be circulated and verified over a network. A blockchain may provide a secure solution for tracking, for example, the ownership and transfer of assets. In a simplified example, a blockchain may provide proof of who owns what at any given point in time and be replicated on hundreds or thousands of computing nodes.

Blockchain structure offers solutions to the dilemma of balancing data, identity, and transaction-based privacy and security. By way of example, security and privacy breaches have occurred, and may continue to happen, within large, often centrally organized entities, such as, for example, big box store retailers, social networks, closed networks, governments and militaries. For consumer facing entities, the privacy and security of customer information, payment information, and transaction histories may be paramount to the success of the business. For closed networks, governments and military networks, the security of data is often directly related to the safety and security of a group of people.

As described herein, blockchain and the related decentralized applications based on blockchain may provide solutions to data security, for example, when using cryptographically-secured encryption as a part of the blockchain used in the particular applications, especially as related to the data parts. Although current systems and networks encrypt data, the decentralizing of various aspects of an information architecture may allow for unintended breaches in currently employed encryption chains and layers as, for example, individual users manipulate and interact with their own data. Using blockchain to hold data, authentication information, and encryption aspects, user data and central repositories may be less vulnerable to data losses or breaches. For example, blockchains may store encrypted information and coded pointers to distributed storage locations that may be spread across distributed computer networks. Such a method may prevent those seeking to access or alter the information in an unauthorized manner from doing so by creating a highly distributed temporal infrastructure which may be impractical to reconstruct or, for example, impossible to reconstruct even if the unauthorized user is able to obtain a portion of the information associated with the blockchain.

In an embodiment of the present invention, the blockchain systems and methods described herein may be used within consensus engines, decentralized architectures and peer-to-peer clients, for example. Similarly, the blockchain systems and methods described herein may be used to facilitate secure communication within the Internet of Things, which may include devices subject to security breaches which arise from the vulnerabilities created by allowing unsecure and vulnerable data exchanges with a device and remote computing resources.

In an embodiment of the present invention, blockchain and AI processing may be used to provide, for example, robust validation techniques to prevent such things as Sybil attacks, for example, and dismiss masquerading hostile entities by providing a secure identity and/or authorization mechanism. For example, a local entity may accept a remote encrypted identity blockchain based on a central authority which may ensure a one-to-one correspondence between an identity and an entity and may even provide a reverse lookup. The identity may be validated either directly or indirectly. In direct validation the local entity queries the central authority to validate the remote identities. In indirect validation the local entity relies on already accepted identities which in turn vouch for the validity of the remote identity in question.

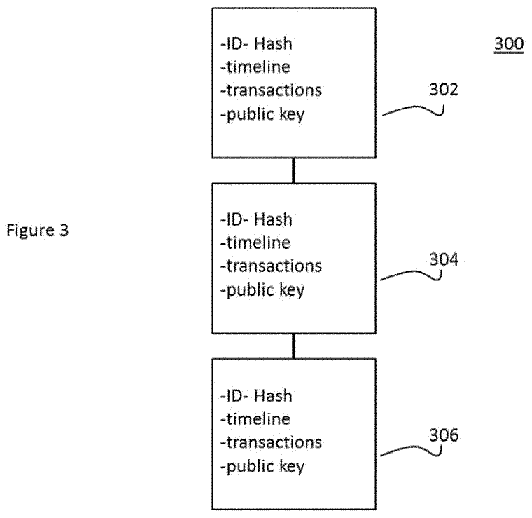

As illustrated in FIG. 3, blockchain 300 is an illustrative example in accordance with at least one embodiment of the invention. Blockchain 300 illustrates a blockchain having 3 blocks, 302, 304, and 306. Block 302 is the first block and is therefore considered to be the genesis block. Each block may include certain information, such as an Identification, or hash, that uniquely identifies the block, a timeline identifying previous blocks (e.g., the hash numbers of previous blocks) in chronological order, transactions to record all transfers between a sender and a receiver, and a public key that identifies at least one sender and at least one receiver. Hash values may be combined into a Merkle tree. The linked blocks therefore form a chain where each link, or block, in the chain uniquely identifies a previous link, or block, by including the hash or the prior link, or block.

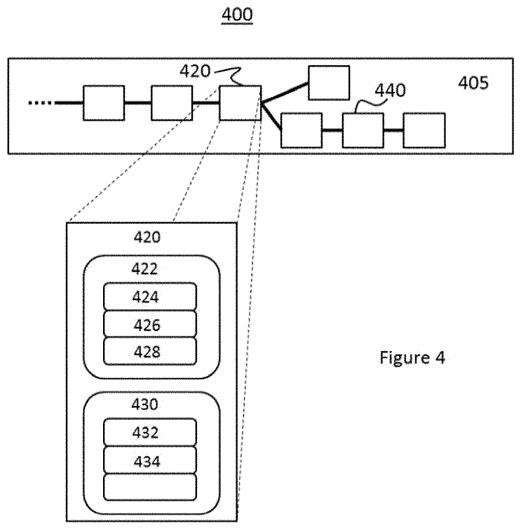

In an embodiment of the present invention, a distributed secure transaction ledger, in the form of a block chain, may be used to communicate data between parties. As illustrated in FIG. 4, a block chain or decentralized secure transaction ledger 405 may be one that is maintained by nodes in a distributed network. Although each block of ledger 405 may contain differentiated information and may have distinct purposes, as illustrated in FIG. 4, block 410 contains a sample communication or message according to embodiments of the present invention.

In an embodiment of the present invention, ledger 405 may be used to send messages between at least two users of a system through, for example, nodes in a network. By way of non-limiting example only, a message in block 420 of the ledger 405 may contain a header 422 and contents 430. The header 422 may comprises at least one block ID 424 for block 420, a block ID 426 of the previous block, and a nonce value 428, an arbitrary number that may be used as a cryptographic hash function. These values and block information may be used in linking blocks together to form a chain.

The contents 430 may comprise one or more messages 432 and may also include other data 434. In an embodiment on the present invention, a message 432 may comprise a unique identifier of the owner/originator/sender of the message. This information may be used for one or more purposes, such as, for example, to identify the owner or sender to provide a way by which a third-party node or nodes that handle and or process the ledger 405. Additionally, the identifier of the owner/sender may be used or linked to an authentication module and/or server associated with using the block chain as a communication channel, or for other actions. Indeed, block 420 may include any number of identifiers which may for example, be used to indicate whether or not the ledger 405 should be directed to a different identifier than the originator of the message 432. As would be appreciated by those skilled in the art, message 432 may include data for processing, which may be obfuscated using, for example, homomorphy transformation.

In an embodiment of the present invention, ledger 440 may be used to send encrypted and secure messages between users through public systems, private/closed systems, or a combination thereof. Similar to the message(s) in block 420, the contents of ledger 440 may comprises one or more messages and may also comprise other encryption/authentication data. In an embodiment of the present invention, a message contained in ledger 440 may comprise a unique identifier of the recipient of the message, which may be the originator of the initial message or another entity. The message may include a unique identifier of the node that submitted the message. Such may be used for one or more purposes. For example, the identifier may help identify who sent the message. Additionally, the identifier may be used or linked to an authentication server/module associated with using the block chain as a communication channel, for performing security, authentication, resolving, or other actions.

In an embodiment of the present invention, the message 432 may include a digitally signed message checksum as way to verify the message. For example, the sender of the message may digitally sign a checksum or hash of the message using his or her private key. A receiving device can verify the integrity of the data by verifying the checksum or hash using the sender's public key. Those having skill in the art shall recognize that other methods for verifying the data's integrity may also be employed herein.

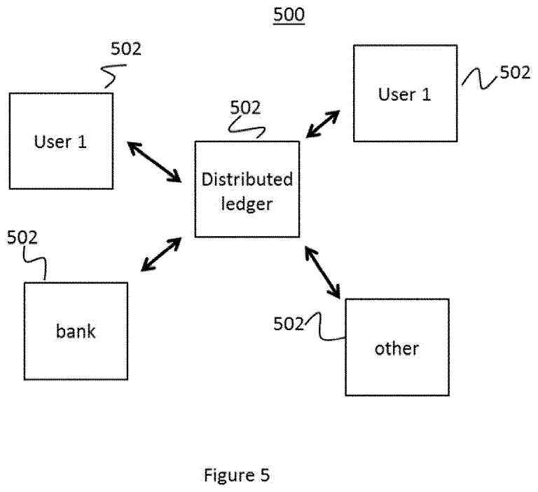

FIG. 5 is a block diagram depicting an example blockchain environment 500, illustrating a simplified example of how a distributed ledger, or blockchain (such as blockchain 405) may be distributed, or replicated, on a network. Environment 500 may include a first user 502, a second user 504, another entity, such as a bank 508, and another third party entity 510 privy to the blockchain 506. The first/second user may be a buyer or seller, based on the transaction. Entities may include, but are not limited to, consumers, bankers, merchants, and investors. Blockchain 506 may be replicated in an agreed-upon manner or in real-time (e.g., after each transaction).

The foundational distinction of the present invention is a risk management program which aligns stakeholder incentives with personalized health outcomes to objectively define & mitigate health risk. This is the only feasible way to deliver sustained control & predictability of Plan performance to employers & health outcomes for Plan members.

The prevailing health insurance risk model is financially focused and by definition, conflicted with the long-term health needs of subscribers.

In a financial risk model, the insurer has no incentive to define & track health outcomes over time, so subscribers have no expectation that system incentives somehow tie in with their own prevailing health status.

Key stakeholder decisions are thus perpetually in conflict with subscriber need for ongoing & unencumbered access to health support. This conflicted model drives stakeholders across the healthcare industry. So regardless of choices to—fully insure, partially or fully self-insure, or use a health captive—the underlying risk management model is a financial one. This is foundational to why employers have no control or predictability of their Plan's performance and likewise, policy holders as to their own health. This country's spiraling health costs and consistently poor outcomes are thus symptomatic of the industry's original design & impotence to mitigate the risks driving health disruption and disease.

The present invention aligns Member and Provider incentives with Member Engagement, Adherence & Health Outcomes. This may also aligns with and drives Captive performance. Financial conflict drives most of the access barriers for Member health support. Removal of access barriers is foundational to engaging members to co-manage/mitigate their own health risk. Engaging Members with their personalized health diagnostics & treatment roadmap is key to their understanding & mitigating the unique drivers & disruptors of their own health.

1. Health Risk Management requires measuring Health Outcomes.

2. Measuring Health Outcomes requires a personalized, precision medical model to identify, measure & track: key biomarkers, lifestyle and environmental factors.

Hence, aligning Health Outcomes with financial incentives requires a 21^{st} century clinical model with the science & tools to replace the 20^{th} century model, with its symptom treatment & chronic disease focus.

This requires a bold shift in moving beyond the prevailing healthcare risk model & delivery system to adopt personalized, precision diagnostics (pharmacogenomics, nutrigenomics, hormones, etc.).

These personal biomarkers point to the unique inflammatory triggers responding to lifestyle & environmental influences. What follows are the metabolic pathway disruptions, leading to chronic disease.

Chronic diseases consume over 90% of our national health spend. Claims data reflects 10% of an employer group plan drives 80% of costs. Aligning Key Metrics & RxToken Incentives provides Financial Alignment Facilitates Biological Alignment, Stronger Engagement Drives Stronger Adherence and Stronger Adherence Drives Stronger Outcomes.

In an embodiment of the present invention, removing system conflict & financially aligning Member/Provider, facilitates voluntary participation, optimizing Member Engagement efforts. Mandated participation is an unnecessary barrier & will have the opposite effect. Member engagement with our personalized, precision health/wellness model will involve Provider health coaching support. Member's potential for incentives should be sufficient to fund offset of: copays, deductibles, share of premium . . . even retirement savings.

Health Providers will be responsible for managing the mitigation of personalized health risk for our Members. Providers will engage Members with education, training & coaching support using our personalized diagnostic & treatment protocols & tools.

These Providers will receive RxTokens as financial incentives for providing services & support which enhance their Members' metrics/incentives.

Providers will also receive RxTokens for standards in operating their practices, including Member ratings of their engagement experience Provider.

In an embodiment of the present invention, Strong Provider/Member Engagement strengthens Adherence & Outcomes. Establish accurate, comprehensive baseline data sets for Member & family medical/symptom profiles, and lifestyle choices/environmental influences. Educate Member how their baseline date drives Incentive Metrics.

Game Theory & A/Machine Learning for continuous feedback loops to measure & enhance quality of all Provider/Member knowledge transfer sessions. Member/Provider use of mobile/remote monitoring (Fitbits, etc.) & communication devices. Social Media/Support Groups/Chat Rms. (Shared Testimonials, etc.).

Members & Providers will be measured against Member's metrics in this component, with other metrics/incentives as warranted, independent of each other. Metrics include: diagnostics, treatments, lifestyle/behavior patterns, cooperation in required feedback loops, use of engagement & health data tracking tools, and other engagement areas TBD. Ongoing biomarker metrics with Member/Provider generated feedback, against baseline data.

Biomarker data reflecting Engagement & Adherence impact.

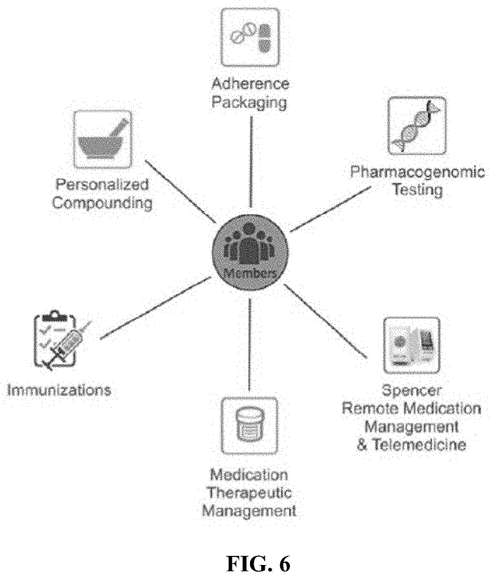

As illustrated in FIG. 6, Using blockchain technology & decentralized apps for smart contracts, the following features will further differentiate the strength & value of our health insurance captive model.

All personalized, HIPAA data will be encrypted and stored in a highly secure, decentralized manner on the blockchain platform designed for this Captive. All Plan Members will be assured ownership of & unrestricted access to both their identity and HIPAA information. Access to Member data will require the authorization of the respective Member. The Captive will not seek to monetize Member/Patient data in any form, for any purpose.

Subject to Member authorization, the Captive will restrict its access to and use of Member/Patient data, exclusively for the ongoing information needs of the Captive's operation.

Additionally, the Captive will engage in clinical studies involving aggregated, de-identified Member data to gain insights into diagnostic and treatment efficacies and in continuous development of its own proprietary clinical protocols in the standard course of managing the personal health risk of its Members/Patients.

In an embodiment of the present invention, all Incentive & transaction related data will be entered on the blockchain platform pursuant to the smart contract parameters re: security, access, validation, transparency, etc.

In an embodiment of the present invention, the Health Risk Model disclosed is a uniquely powerful differentiator. For the first time, the financial interests of Providers & Members will both be in alignment with those of the Insurance Plan.

The research science driving personalized, precision Diagnostics will continue to evolve and become ever more relevant to our clinical & risk models. Continuous refinement of Diagnostic & Treatment Protocols is critical to scaling the Captive. We will retain qualified scientists/researchers for management of this critical function.

The present invention may use AI/Machine Learning to strengthen Diagnostic/Treatment Protocols & reduce risks for accelerated scaling. We will also leverage AI-resourced Protocols for stronger Provider/Member Engagement, as well as Adherence and Outcomes. Additionally, Game Theory/Gamification will be developed to resource Engagement & Adherence to optimize Outcomes.

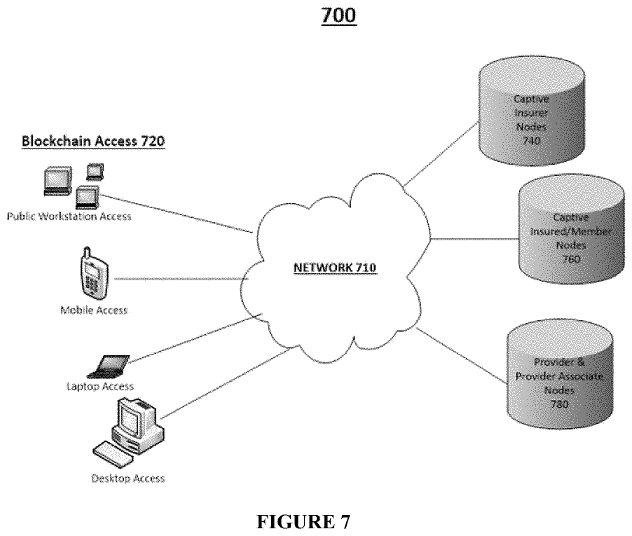

FIG. 7 is an example of a blockchain environment 700 which shows how the invention herein may be implemented on a blockchain. FIG. 7 shows that insurers may access and write to a blockchain network via Captive Insurer Nodes 740 only to areas of the blockchain that they are allowed to. FIG. 7 also shows that insured members may access and write to a blockchain network via Captive Insured/Member Nodes 760 only to areas of the blockchain that they are allowed to. Also, health providers and their associates can access and write to a blockchain network via Provider & Provider Associate Nodes 780 only to areas of the blockchain that they are allowed to.

Also shown in FIG. 7 is that an individual, whether an insurer, member, or provider, may gain individual access to the blockchain via a computing device under Blockchain Access 720, or the like. It is also established in this figure that different members of a blockchain may be connected to each other via Network 710.

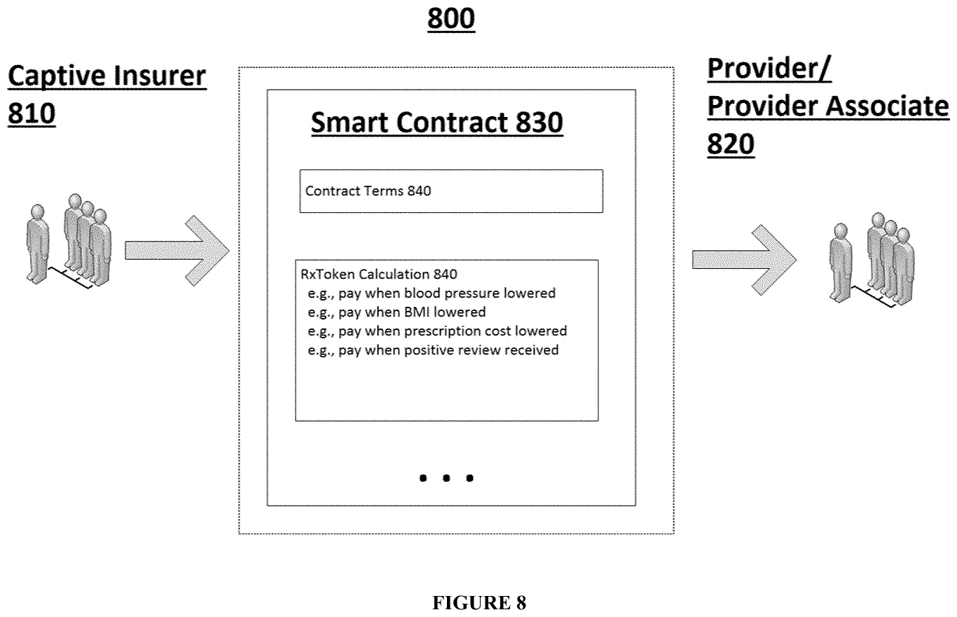

FIG. 8 is an example of a smart contract, on a blockchain, which may be utilized by the invention herein. Captive Insurer 810 may come into an agreement with Provider/Provider Associate 820 in order to create a Smart Contract 830 on the blockchain. As shown in this figure, the contract terms may be saved, in the smart contract, under Contract Terms 840. The RxToken Calculation 840 represents the code which may be stored in the smart contract that executes when a condition is met. For example, an insurer and a provider may contract for the provider to automatically be awarded a blockchain enabled currency RxToken when an insured member's blood pressure is lowered due to the provider's assistance to the member. This transfer of value, RxToken, over the blockchain, would automatically execute from the code saved within the smart contract.

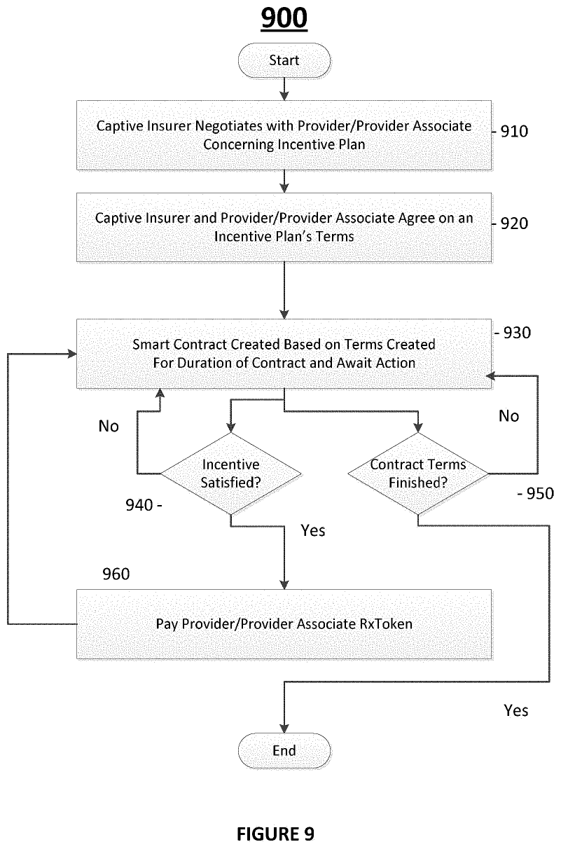

FIG. 9 shows an example process flow 900. At 910, a captive insurer may negotiate with a provider or provider Associate concerning incentive plan where the provider would receive a certain number of RxTokens through the blockchain as an incentive when a certain condition is met (e.g., member BMI is lowered). Step 920 represents the agreement of terms of the incentive contract between the parties. At 930, the terms of the agreement between the insured and the provider are saved on a smart contract, where the payment of RxTokens are paid to the provider automatically based on the condition of the term which the provider must meet.

At decision 940, the smart contract determines if a condition term is satisfied (e.g., provider receives 10 positive reviews from members) and automatically pays the number of agreed upon RxTokens on the blockchain to the provider. If the condition is not satisfied, then the smart contract will wait until any of the conditions coded into the smart contract are satisfied. At decision 950, the smart contract checks to see if the contract duration is still valid, if so, then the smart contract continues to wait for a satisfying condition to payout RxTokens, if not, then the smart contract ends itself and the parties may no longer perform the terms of the contract on that specific smart contract.

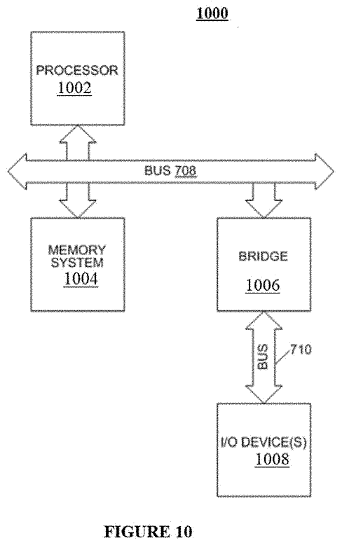

FIG. 10 is an example of a simplified functional block diagram of a computer system 1000. The functional descriptions of the present invention can be implemented in hardware, software or some combination thereof. For example, a recommendation engine and an integration engine of the present invention can be implemented using a computer system.

As shown in FIG. 10, the computer system 1000 includes a processor 1002, a memory system 1004 and one or more input/output (I/O) devices 1008 in communication by a communication ‘fabric’. The communication fabric can be implemented in a variety of ways and may include one or more computer buses 708, 710 and/or bridge and/or router devices 712 as shown in FIG. 7. The I/O devices 1008 can include network adapters and/or mass storage devices from which the computer system 1000 can send and receive data for generating and transmitting advertisements with endorsements and associated news. The computer system 1000 may be in communication with the Internet via the I/O devices 1008.

Those of ordinary skill in the art will recognize that many modifications and variations of the present invention may be implemented without departing from the spirit or scope of the invention. Thus, it is intended that the present invention cover the modification and variations of this invention provided they come within the scope of the appended claims and their equivalents.

The various illustrative logics, logical blocks, modules, and engines, described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Further, the steps and/or actions of a method or algorithm described in connection with the aspects disclosed herein may be embodied directly in hardware, in a software module executed by a processor, or in a combination of the two. A software module may reside in RAM memory, flash memory, ROM memory, EPROM memory, EEPROM memory, registers, a hard disk, a removable disk, a CD-ROM, or any other form of storage medium known in the art. An exemplary storage medium may be coupled to the processor, such that the processor can read information from, and write information to, the storage medium. In the alternative, the storage medium may be integral to the processor. Further, in some aspects, the processor and the storage medium may reside in an ASIC. Additionally, the ASIC may reside in a user terminal. In the alternative, the processor and the storage medium may reside as discrete components in a user terminal. Additionally, in some aspects, the steps and/or actions of a method or algorithm may reside as one or any combination or set of instructions on a machine readable medium and/or computer readable medium.

Those of skill in the art will appreciate that the herein described apparatuses, engines, devices, systems and methods are susceptible to various modifications and alternative constructions. There is no intention to limit the scope of the invention to the specific constructions described herein. Rather, the herein described systems and methods are intended to cover all modifications, alternative constructions, and equivalents falling within the scope and spirit of the disclosure, any appended claims and any equivalents thereto.

In the foregoing detailed description, it may be that various features are grouped together in individual embodiments for the purpose of brevity in the disclosure. This method of disclosure is not to be interpreted as reflecting an intention that any subsequently claimed embodiments require more features than are expressly recited.

Further, the descriptions of the disclosure are provided to enable any person skilled in the art to make or use the disclosed embodiments. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but rather is to be accorded the widest scope consistent with the principles and novel features disclosed herein.

1. A non-transitory computer-readable storage device having contents adapted to cause a programmed computer system to perform operations comprising:
storing, on a blockchain network, a smart contract where a captive insurer agrees to transfer a certain amount of RxTokens to a health provider, or a health provider associate, through the blockchain network, conditioned on at least one term agreed upon by both the captive insurer and the health provider or the health provider associate;
transferring, on the blockchain network, through the smart contract, RxTokens, automatically, when any of the terms agreed upon are met, to the health provider or the health provider associate;
wherein the number of RxTokens transferred is dependent on the contract term satisfied, and an agreed upon number of RxTokens to be transferred upon satisfaction of the that contract term;
recording, on the blockchain network, through the smart contract, on a ledger block, the number of RxTokens transferred to the health provider or the health provider associate, and the agreed upon term satisfied;
wherein the ledger block is viewable to authorized nodes.

2. The computer-readable storage device of claim 1, wherein the contract term may be satisfied by receiving input from a mobile/remote monitoring & communication device, such as a Fitbit.

3. The computer-readable storage device of claim 1, wherein the contract term may be satisfied by receiving precision diagnostics inputs such as pharmacogenomics.

4. The computer-readable storage device of claim 1, wherein the contract term result may be intended to provide a health benefit to the captive insurer's insured.

5. The computer-readable storage device of claim 1, wherein the contract term result may be intended to provide a cost savings benefit to the captive insurer's insured.

6. A method comprising:
storing, on a blockchain network, a smart contract where a captive insurer agrees to transfer a certain amount of RxTokens to a health provider, or a health provider associate, through the blockchain network, conditioned on at least one term agreed upon by both the captive insurer and the health provider or the health provider associate;
transferring, on the blockchain network, through the smart contract, RxTokens, automatically, when any of the terms agreed upon are met, to the health provider or the health provider associate;
wherein the number of RxTokens transferred is dependent on the contract term satisfied, and an agreed upon number of RxTokens to be transferred upon satisfaction of the that contract term;
recording, on the blockchain network, through the smart contract, on a ledger block, the number of RxTokens transferred to the health provider or the health provider associate, and the agreed upon term satisfied;
wherein the ledger block is viewable to authorized nodes.

7. The method of claim 6, wherein the contract term may be satisfied by receiving input from a mobile/remote monitoring & communication device, such as a Fitbit.

8. The method of claim 6, wherein the contract term result may be intended to provide a health benefit to the captive insurer's insured.

9. The method of claim 6, wherein the contract term result may be intended to provide a cost savings benefit to the captive insurer's insured.

10. The method of claim 6, wherein the contract term may be satisfied by receiving precision diagnostics inputs such as pharmacogenomics.
